(19) United States
(12) Patent Application Publication
(10) Pub. No.: US 2017/0115308 A1
(43) Pub. Date: Apr. 27, 2017

(54) ASSAY TO MEASURE EFFICACY OF CDK8/19 INHIBITORS

(71) Applicant: SENEX BIOTECHNOLOGY, INC., Columbia, SC (US)

(72) Inventor: Igor B. Roninson, Lexington, SC (US)

(73) Assignee: SENEX BIOTECHNOLOGY, INC., Columbia, SC (US)

(21) Appl. No.: 14/955,291

(22) Filed: Dec. 1, 2015

Related U.S. Application Data
(60) Provisional application No. 62/079,767, filed on Nov. 14, 2014.

(51) Int. Cl. G01N 33/68

(52) U.S. Cl. CPC G01N 33/6872; G01N 2333/47; G01N 2440/14; G01N 2500/10

(57) ABSTRACT

The invention provides a method for determining the efficacy of a small molecule for inhibiting cyclin-dependent kinase 8 (CDK8) and/or cyclin-dependent kinase 19 (CDK19), using STAT1 phosphorylation as a PD marker.

### PRIORITY DATA

This application claims the benefit of priority to U.S. Provisional Application No. 62/079,767, filed Nov. 14, 2014.

### BACKGROUND OF THE INVENTION

Field of the Invention

The invention relates to the development of CDK8/19 inhibitors. More particularly, the invention relates to measuring the pharmacodynamics of CDK8/19 inhibitors.

Summary of the Related Art

Drug development is a slow and costly process. To accelerate the development of molecularly targeted drugs, various tissue-based approaches have become widely used to perform pharmacodynamic (PD) studies in early phase clinical trials (Ang et al., 2012). These approaches measure drug response using mechanistic molecular markers of the drug's target inhibition. The pharmacokinetic (PK)/PD modelling is also used in preclinical studies to elucidate the optimal dosing and scheduling for subsequent clinical development (Tate et al., 2014).

Cyclin-dependent kinase 8 (CDK8), along with its closely related paralog CDK19, has recently emerged as an exciting new target for the treatment of cancer (Porter et al., 2012) (U.S. Pat. No. 8,598,344; US Patent Publication 20140038958; U.S. Application No. 62/010,074; PCT/US2013/67990; PCT/US2014/18678) and other diseases, in particular those associated with NFκB activity (WO2013/040153). CDK8 (ubiquitously expressed), along with CDK19 (which is expressed in only a subset of tissues), is an oncogenic transcription-regulating kinase (Xu and Ji, 2011; Galbraith et al., 2010; Firestein and Hahn, 2009), a subunit of a regulatory module of the Mediator complex that connects transcription-initiating factors with RNA Pol II (Galbraith et al., 2010). In contrast to better-known members of the CDK family (such as CDK1, CDK2 and CDK4/6), CDK8/19 play no general role in cell cycle progression, and their binding partner, Cyclin C, is expressed relatively uniformly through different phases of the cell cycle. CDK8 knockout in embryonic stem cells prevents embryonic development (Westerling et al., 2007), due to the essential role of CDK8 in the pluripotent stem cell phenotype (Adler et al., 2012) but CDK8 depletion does not inhibit the growth of normal cells (Westerling et al., 2007; Firestein et al., 2008). Furthermore, CDK8/19 kinase inhibitors are neither cytotoxic nor cytostatic to normal cells or to most of the tested tumor cell types (Porter et al., 2012). The lack of effect of CDK8 inhibition on the growth of most cell types is due to the fact that CDK8 does not appear to be involved in the regulation of constitutively expressed genes but functions primarily at physiological transitions, by activating gene expression in response to specific signals through enabling the elongation of newly initiated transcription (Donner et al., 2010; Galbraith et al., 2013).

The role of CDK8 in cancer is due to its unique function as a regulator of several transcriptional programs involved in carcinogenesis (Xu and Ji, 2011) and chemotherapeutic drug response (Porter et al., 2012). CDK8 has been implicated in melanomogenesis (Kapoor et al., 2010) and identified as an oncogene in colon cancer (Firestein et al., 2008), where the CDK8 gene is amplified in ˜50% of cancers. We have previously reported the first selective small-molecule inhibitors of CDK8/19 (Porter et al., 2012) (U.S. Pat. No. 8,598,344; US Patent Publication 20140038958) and demonstrated that these inhibitors have chemopotentiating, chemopreventive and anti-metastatic activities (US Patent Publication 20140038958) and that CDK8/19 inhibitors directly suppress the growth of androgen receptor-dependent prostate cancers (PCT/US2013/67990) and estrogen receptor-dependent breast cancers (U.S. Application No. 62/010,074).

The development of CDK8/19 inhibitors could be greatly facilitated by the availability of mechanistically linked PD markers, i.e. proteins, the phosphorylation of which would be decreased upon CDK8/19 inhibition. An especially valuable PD marker would be a specific CDK8/19 kinase target, the phosphorylation of which is directly involved in the regulation of transcription by CDK8/19, as changes in phosphorylation of such protein would have direct functional consequences.

Identification of CDK8/19 phosphorylation targets that can be used as PD markers proved to be elusive since such proteins need to be phosphorylated primarily or exclusively by CDK8/19, as their phosphorylation by other kinases would make it difficult or impossible to detect a decrease in phosphorylation upon CDK8/19 inhibition. In vitro studies have identified several proteins that can be phosphorylated by CDK8 (few studies have been conducted with CDK19). These phosphorylation substrates include CDK8 itself and the C-terminal domain (CTD) of RNA polymerase II (pol II) (Liao et al., 1995), TFIIH (Akoulitchev et al., 2000) and Histone H3, which is phosphorylated by CDK8 at S10 (Knuesel et al., 2009). However, there has been no evidence that CDK8/19 inhibition leads to decreased global phosphorylation of these targets in different cell types.

An important transcriptional regulator has been recently reported to be phosphorylated by CDK8. This protein is STAT (signal transducer and activator of transcription) 1. STAT1 is activated by interferons (IFNs) and transmits signals from the cell surface to the nucleus. STAT1 activity is regulated by phosphorylation of tyrosine701 (Y701), which is carried out by JAK and certain other tyrosine kinases and which enables STAT1 dimerization and nuclear translocation. Once bound to chromatin (Sadzak et al., 2008), STAT1 is phosphorylated within its transactivation domain at serine727 (S727). S727 phosphorylation was found to maximize the transcriptional activity of STAT1 (Lodige et al., 2005). Phospho-S727 STAT1 was shown to have pro-carcinogenic and anti-apoptotic activity (Timofeeva et al., 2006).

Several kinases have been identified as responsible for STAT1 S727 phosphorylation, including p38 MAPK (Kovarik et al., 1999), CAMKII (Nair et al., 2002), PKC delta (Deb et al., 2003) and CK2 (Timofeeva et al., 2006). Two recent articles have shown that CDK8 also acts as a STAT1 S727 kinase; both of these articles demonstrated the ability of CDK8 to phosphorylate STAT1 at S727 in vitro but they reached somewhat different conclusions about the role of CDK8 in STAT1 phosphorylation in vivo. In the first article (Bancerek et al., 2013), shRNA knockdown of CDK8 or of Cyclin C (the binding partner of both CDK8 and CDK19) inhibited IFN-gamma induced STAT1 S727 phosphorylation in mouse embryo fibroblasts. CDK8 knockdown, however, had no effect on the basal STAT1 S727 phosphorylation (i.e. without the addition of IFN-gamma). Furthermore, a pan-CDK inhibitor flavopiridol failed to inhibit the enhancement of STAT1 S727 phosphorylation by LPS or anisomycin, leading the authors to conclude that CDK8 phosphorylates STAT1 only under the conditions of IFN-gamma stimulation (Bancerek et al., 2013). The second article (Putz et al., 2013) analyzed STAT1 S727 phosphorylation in mouse natural killer (NK) cells, concluding that in these cells, in contrast to the fibroblasts, CDK8 knockdown by shRNA inhibited constitutive basal STAT1 S727 phosphorylation. Notably, while both of the above studies demonstrated that CDK8 phosphorylates STAT1 S727 in vitro and that CDK8 knockdown affects STAT1 S727 phosphorylation in vivo, they did not demonstrate that this in vivo effect involved direct interaction between CDK8 and STAT1. Furthermore, these articles suggest that a decrease in STAT1 S727 phosphorylation could be used as a marker of CDK8 inhibition but only in the cells or organisms treated with IFN-gamma or in isolated NK cells, and that constitutive basal STAT1 S727 phosphorylation would be unaffected by CD8/19 inhibition other than in NK cells.

There is, therefore, a need to identify a specific CDK8/19 kinase target, the phosphorylation of which is directly involved in the regulation of transcription by CDK8/19.

### BRIEF SUMMARY OF THE INVENTION

The invention provides a specific CDK8/19 kinase target, the phosphorylation of which is directly involved in the regulation of transcription by CDK8/19.

The present inventors have surprisingly found that selective small-molecule inhibitors of CDK8/19 inhibit constitutive basal STAT1 S727 phosphorylation in many different types of human cells (which are not NK). The present inventors have validated this result through double shRNA knockdown of CDK8 and CDK19 and demonstrated that STAT1 S727 phosphorylation by CDK8 is involved in the regulation of NFκB activity, a medically important function of CDK8. These results identify STAT1 S727 phosphorylation as a pharmacodynamic marker of CDK8/19 inhibition in different human cell types, suitable for use in preclinical and clinical development of CDK8/19 inhibitors.

The invention provides a method for determining the efficacy of a small molecule for inhibiting cyclin-dependent kinase 8 (CDK8) and/or cyclin-dependent kinase 19 (CDK19). The method according to the invention comprises obtaining, from a subject that has been administered the small molecule and has not been administered interferon-γ, a tissue sample or a sample derived from a tissue sample, other than natural killer cells, assaying the sample for signal transducer and activator of transcription protein 1 (STAT1) that is phosphorylated at serine 727 (pSTAT1), quantitating the amount of pSTAT1 in the sample, comparing the amount of pSTAT1 in the sample with an amount of pSTAT1 in a standard, and determining that the small molecule has efficacy for inhibiting CDK8/19 if the amount of pSTAT1 in the sample is statistically significantly lower than the amount of pSTAT1 in the standard. In another embodiment of the invention, the small molecule is administered to animal cells in cell culture. In some embodiments, the animal cells are human cells.

### BRIEF DESCRIPTION OF THE DRAWINGS

FIG. 1A shows results of immunoblotting for phospho-STAT1 S727 (pSTAT1), total STAT1 (tSTAT1), CDK8, CDK19 and GAPDH (loading control), as a measure of the effect of different concentrations of Senexin B (6 hr treatment) on STAT1 S727 phosphorylation in derivatives of LNCaP-LN3 prostate cancer cell line transduced with lentiviral vectors expressing shRNAs against CDK8 and CDK19 (double knockdown, DK) or the corresponding insert-free pLKO.1 and pHLB vectors (double vector, DV). FIG. 1B shows pSTAT1/tSTAT1 ratio, as determined by densitometry, normalized to control DV cells.

FIG. 2A shows the time course of the inhibition of STAT1 S727 phosphorylation in these cells using 5 μM of Senexin B. Here the effect was also noticeable after 15 min and very strong after 1 hr of treatment. FIG. 2B shows concentration-dependent inhibition of STAT1 S727 phosphorylation in these cells, after 24 hr treatment with Senexin B. FIG. 2C shows concentration-dependent inhibition of STAT1 S727 phosphorylation in these cells, after 24 hr treatment with Cortistatin A. FIG. 2D shows results of the same analysis using a chemically unrelated CDK8/19 inhibitor, 16-didehydro-cortistatin A, an equipotent analog of a steroidal alkaloid cortistatin A (Porter et al., 2012). FIG. 2 E shows that this compound inhibited STAT1 S727 phosphorylation similarly to Senexin B, further confirming that Senexin B exerts its effect via CDK8/19 inhibition.

FIG. 3A shows effects of 24-hr treatment with 5 μM Senexin B on STAT1 S727 phosphorylation in MCF7 breast cancer cells, as detected with two different antibodies against phospho-S727 STAT1. FIG. 3B shows effects of 24-hr treatment with the indicated concentrations of Senexin B on STAT1 S727 phosphorylation in MCF7 breast cancer cells. FIG. 3C shows densitometry of phospho-S727 STAT1/total STAT1 ratio from the experiment in (B). FIG. 3D shows effects of treatment with Senexin B (1 μM and 5 μM) for different periods of time on STAT1 S727 phosphorylation in SKBR3 breast cancer cells. FIG. 3E shows densitometry of phospho-S727 STAT1/total STAT1 ratio from the experiment in (D). FIG. 3F shows effects of treatment with Senexin B (1 μM and 5 μM) for different periods of time on STAT1 S727 phosphorylation in BT474 breast cancer cells. FIG. 3G shows effects of treatment with Senexin B (5 μM) for 6 hrs on STAT1 S727 phosphorylation in MDA-MB-361 breast cancer cells.

FIG. 4A shows results of staining with phospho-STAT1 S727 primary antibody (1:1,000 dilution), followed by GFP-conjugated secondary antibody; merged GFP fluorescence and DIC imaging are shown. FIG. 4B shows results of staining with total-STAT1 S727 primary antibody (1:400 dilution), followed by GFP-conjugated secondary antibody.

FIG. 5 shows chromatin immunoprecipitation (ChIP) analysis of 293 cells pre-treated with 5 mM Senexin A or carrier for one hour, at which point either TNFα at 10 ng/ml or carrier was added for 30 min. Sonicated chromatin was immunoprecipitated with the antibodies against the indicated proteins. The ChIP preps were used for quantitative real-time PCR amplification of different segments of the indicated genes (X-axes show the positions of the tested segments relative to transcription start sites). The results of this analysis for all four conditions (blue, control; red, Senexin A; green, TNFα; purple, TNFα plus Senexin A) are shown, with the genomic positions of the PCR primers diagrammed at the top of the figure.

FIG. 6A shows effects of treatment with Senexin A (5 μM) for different periods of time on STAT1 S727 phosphorylation in human embryonic kidney (HEK) 293 cells. FIG. 6B shows immunoblotting analysis of total RNA polymerase II (Pol II), and its S2P and SSP C-terminal domain phosphorylated forms, in HEK 293 cells treated under the same conditions as in FIG. 5. Results are shown for three biological replicates.

FIG. 7A shows immunofluorescence analysis of CDK8 and phospho-STAT1 expression in normal lung tissues. FIG. 7B shows immunofluorescence analysis of CDK8 and phospho-STAT1 expression in lung adenocarcinoma tissues. Images (left to right) show staining with DAPI (a nuclear dye), anti-CDK8 primary antibody followed by Alexa555-conjugated secondary antibody, anti-phospho-STAT1 S727 primary antibody followed by Cy5-conjugated secondary antibody, and merged Alexa555 and Cy5 imaging.

### DETAILED DESCRIPTION OF THE PREFERRED EMBODIMENTS

The invention provides a specific CDK8/19 kinase target, the phosphorylation of which is directly involved in the regulation of transcription by CDK8/19.

The present inventors have surprisingly found that selective small-molecule inhibitors of CDK8/19 inhibit constitutive basal STAT1 S727 phosphorylation in many different types of human cells (which are not NK). The present inventors have validated this result through double shRNA knockdown of CDK8 and CDK19 and demonstrated that CDK8 and STAT1 interact in vivo and that STAT1 S727 phosphorylation by CDK8 is involved in the regulation of NFκB activity, a medically important function of CDK8. These results identify STAT1 S727 phosphorylation as a pharmacodynamic marker of CDK8/19 inhibition in different human cell types, suitable for use in preclinical and clinical development of CDK8/19 inhibitors.

The invention provides a method for determining the efficacy of a small molecule for inhibiting CDK8 and/or CDK19. The method according to the invention comprises obtaining, from a subject that has been administered the small molecule and has not been administered interferon-γ, a tissue sample or a sample derived from a tissue sample, other than natural killer cells, assaying the sample for signal transducer and activator of transcription protein 1 (STAT1) that is phosphorylated at serine 727 (pSTAT1), quantitating the amount of pSTAT1 in the sample, comparing the amount of pSTAT1 in the sample with an amount of pSTAT1 in a standard, and determining that the small molecule has efficacy for inhibiting CDK8/19 if the amount of pSTAT1 in the sample is statistically significantly lower than the amount of pSTAT1 in the standard.

In another embodiment of the invention, the small molecule is administered to animal cells in cell culture. In some embodiments, the animal cells are human cells.

For purposes of the invention, a “small molecule” means a chemical having a formula weight of less than about 900 Daltons. For purposes of the invention, “efficacy” means the ability of the small molecule to reduce one or more biochemical function of CDK8/19. One such biochemical function of CDK8/19 is phosphorylation of STAT1 at serine 727. For purposes of the invention, “STAT1” means signal transducer and activator of transcription protein 1 that is not phosphorylated at serine 727. In contrast “pSTAT1” means STAT1 protein that is phosphorylated at serine 727.

For purposes of the invention, a “tissue sample” is a substance either in the body of the subject, or taken from the body of the subject, that contains one or more types of cells. For purposes of the invention, the term “other than natural killer cells” means that the tissue sample either contains no NK cells, or contains sufficiently few NK cells so as not to provide a false positive assay result. Preferably, such tissue sample should contain no more than 30% NK cells, most preferably no more than 10% NK cells. For purposes of the invention, a “sample derived from a tissue sample” means a substance that has been made through manipulation of a tissue sample. Some such derived samples include, without limitation, whole cell lysates and one or more nucleoplasmic fraction that has been separated from a chromatin fraction. Two such procedures for obtaining such fractions are taught by Aygun et al. (2008) and Lavallee-Adam et al. (2013) In some embodiments, the sample contains or is derived from one or more cell types selected from the group consisting of cancer cells, PBMCs, hair follicle cells, platelets, skin cells, and oral buccal cells. In some embodiments, the subject is a mammal. In some embodiments, the mammal is a human. In some embodiments, the mammal is a rodent.

In some embodiments, the sample is assayed from about 30 minutes to about 72 hours after the subject has been administered the small molecule. In some embodiments, the sample is in situ. In some embodiments, the assay is selected from the group consisting of immunoblotting, ELISA, flow cytometry, immunofluorescence, immunohistochemistry and non-antibody based protein detection methods. In some embodiments, the assay uses monospecific polyclonal antibodies that bind pSTAT1, but do not bind unphosphorylated STAT1. In some embodiments, the assay uses monoclonal antibodies that bind pSTAT1, but do not bind unphosphorylated STAT1. In some embodiments, a monospecific polyclonal antibody or monoclonal antibody that binds unphosphorylated STAT1 is used as a normalization control. Non-antibody based protein detection methods include, without limitation, those taught by Gonzalez-Gonzalez et al. (2012) and Gremel et al. (2013).

In some embodiments, the standard is a tissue sample obtained from the subject prior to administration of the small molecule. In some embodiments, the standard is derived from the tissue sample and includes, without limitation, whole cell lysates and one or more nucleoplasmic fraction separated from a chromatin fraction.

The following examples are intended to further illustrate certain preferred embodiments of the invention and are not to be construed as limiting the scope of the invention.

### Example 1

### CDK8/19 Inhibition Decreases STAT1 S727 Phosphorylation in Different Human Cell Lines

STAT1 S727 phosphorylation was analyzed by immunoblotting using the following primary antibodies: Phospho-Stat1 (Ser727) (D3B7) Rabbit mAb #8826 (used at 1:1000 dilution) and Rabbit Ab #9177 (used at 1:500 dilution) from Cell Signaling Technology (Danvers, Mass.), and total-STAT1 Stat1 p84/p91 Antibody (M-22): sc-592 from Santa Cruz Biotechnology (Dallas, Tex.), used at 1:500 dilution. As a loading control an antibody against a housekeeping protein GAPDH (6C5 mouse monoclonal antibody from Abcam), was used at 1:1000 dilution.

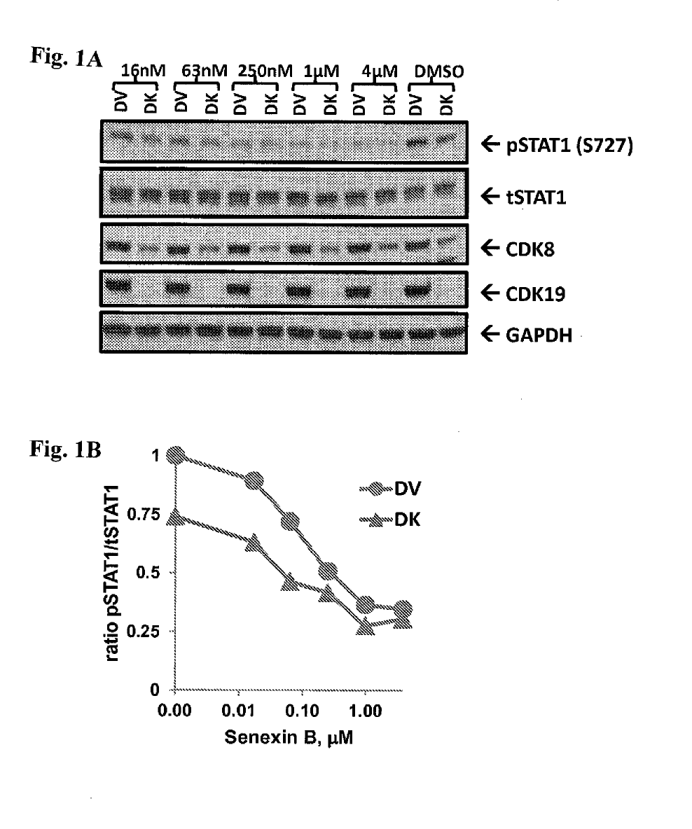

In the experiment shown in FIG. 1, human LNCaP-LN3 cells, an in vivo-derived metastatic variant of LNCaP prostate cancer cells were used. We have compared STAT1 S727 phosphorylation in two derivatives of LNCaP-LN3, one with shRNA knockdown of both CDK8 and CDK19 (CDK8 shRNA was expressed from lentiviral vector PHLB carrying blasticidin resistance marker; CDK19 shRNA was expressed from lentiviral vector pLKO.1 carrying puromycin resistance marker) (DK, double knockdown), and in control cells transduced with insert-free PHLB and pLK0.1 vectors (DV, double vector). Cells were treated for 5 hrs with different concentrations of CDK8/19-selective inhibitor Senexin B (a.k.a. SNX2-1-165) or DMSO control, before being lysed for immunoblotting analysis. The immunoblotting for CDK8 (sc-1521 goat polyclonal antibody from Santa Cruz) and CDK19 (a27580 rabbit polyclonal antibody from Sigma) showed that CDK8 protein level was significantly decreased and CDK19 protein was almost completely abolished in DK relative to DV cells (FIG. 1A). The immunoblotting for phospho-STAT1 S727 (pSTAT1) and total STAT1 (tSTAT1) is shown in FIG. 1A, and image quantitation for the ratio of pSTAT1/tSTAT1 is shown in FIG. 1B. CDK8/19 knockdown decreases basal STAT1 S727 phosphorylation, as does Senexin B, even at the lowest concentrations (starting at 16 nM). With increasing Senexin B concentrations (starting from 1 μM), STAT1 S727 phosphorylation plateaus at a low level, which was the same in DV and DK cells (FIG. 1B), confirming that Senexin B exerts its effect via CDK8/19 inhibition.

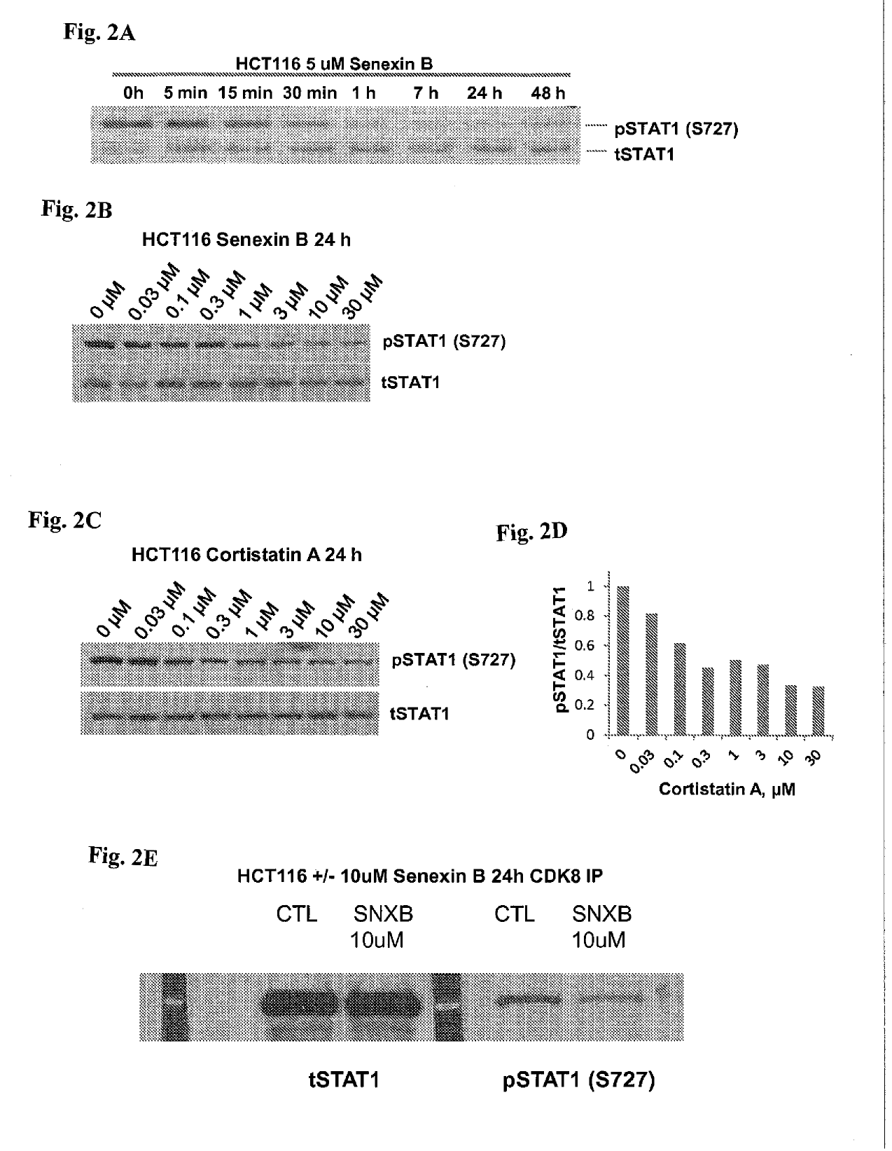

In the experiments shown in FIG. 2, this analysis was continued in HCT116 human colon carcinoma cells. FIG. 2A shows the time course of the inhibition of STAT1 S727 phosphorylation in these cells using 5 μM of Senexin B. Here the effect was also noticeable after 15 min and very strong after 1 hr of treatment. FIG. 2B shows concentration-dependent inhibition of STAT1 S727 phosphorylation in these cells, after 24 hr treatment with Senexin B. FIG. 2C shows concentration-dependent inhibition of STAT1 S727 phosphorylation in these cells, after 24 hr treatment with Cortistatin A. FIG. 2D shows results of the same analysis using a chemically unrelated CDK8/19 inhibitor, 16-didehydro-cortistatin A, an equipotent analog of a steroidal alkaloid cortistatin A (Porter et al., 2012). FIG. 2 E shows that this compound inhibited STAT1 S727 phosphorylation similarly to Senexin B, further confirming that Senexin B exerts its effect via CDK8/19 inhibition.

To determine whether the effect of CDK8/19 inhibitors on STAT1 S727 phosphorylation involved direct interaction between CDK8 and STAT1, we have asked if STAT1 and CDK8 would co-immunoprecipitate, and if so, whether CDK8/19 kinase inhibition would affect STAT1 phosphorylation in complexes with CDK8. FIG. 2F shows an experiment where CDK8 was immunoprecipitated as described (Porter et al., 2012) from HCT116 cells treated for 24 hrs with vehicle control or 10 μM Senexin B, and then immunoblotted for either total STAT1 or phospho-STAT1 S727. This experiment shows that STAT1 and CDK8 interact in vivo, and that CDK8 kinase inhibition does not affect the binding of total STAT1 but inhibits its phosphorylation at S727. Hence, STAT1 phosphorylation in vivo is a direct effect of CDK8.

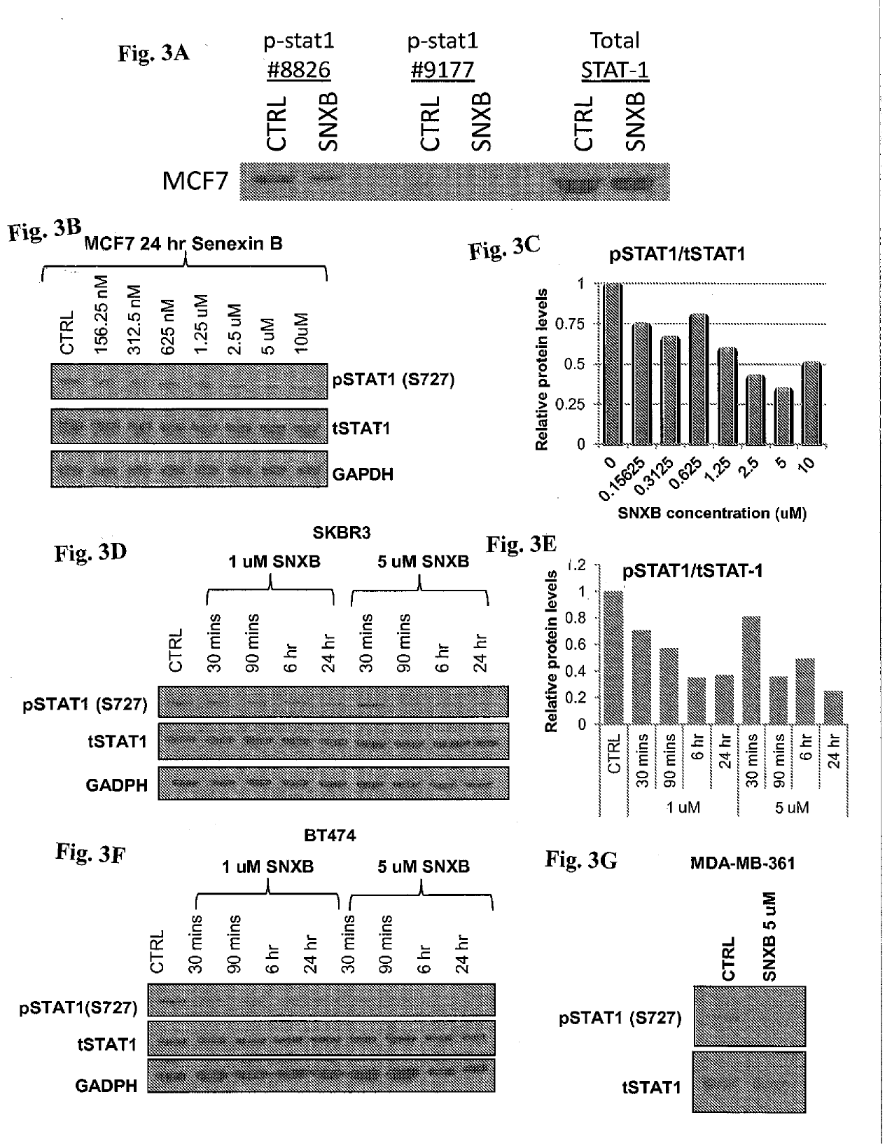

FIG. 3 illustrates the inhibition of basal STAT1 S727 phosphorylation by CDK8/19 inhibitors in human breast cancer cell lines. FIG. 3A shows inhibition of STAT1 S727 phosphorylation in MCF7 human breast cancer cells (ER+ HER2−), after 24 hr treatment with 5 μM Senexin B, as demonstrated with two different antibodies, rabbit mAb #8826 and rabbit Ab #9177. FIG. 3B,C shows concentration-dependent inhibition of STAT1 S727 phosphorylation in MCF7 human breast cancer cells (ER+ HER2−), after 24 hr treatment with Senexin B. FIG. 3D,E shows the time course of the inhibition of STAT1 S727 phosphorylation in SKBR3 human breast cancer cells (ER− HER2+), treated for different periods of time with 1 μM or 5 μM Senexin B, with strong inhibition observed even after 30 min of treatment. FIG. 3F shows the same experiment conducted in BT474 human breast cancer cells (ER+ HER2+), with similar results to SKBR3. FIG. 3G shows that 6 hr treatment with 5 μM Senexin B inhibits STAT1 S727 phosphorylation in another ER+ HER2+ human breast cancer cell line, MDA-MB-361.

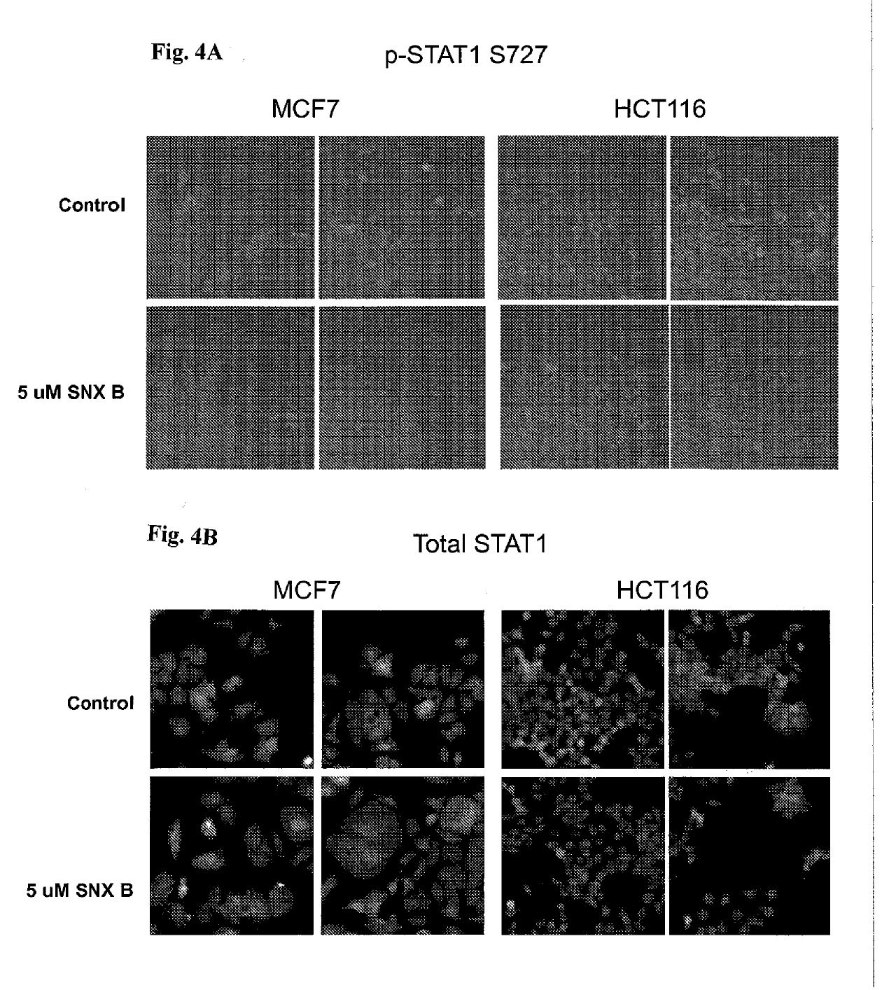

FIG. 4 shows that the inhibition of STAT1 S727 phosphorylation by Senexin B in MCF7 and HCT116 cells can be observed in situ, by immunofluorescence staining of cells with phospho-STAT1 S727 antibody. In contrast, Senexin B shows no apparent effect on staining of the same cells with the antibody against total STAT1. Notably, while total STAT1 is expressed both in the nucleus and the cytoplasm, phospho-STAT1 staining is specific for the nucleus. Taken together, these results demonstrate that CDK8/19 inhibition decreases constitutive basal STAT1 S727 phosphorylation in all the tested human cell lines of different origins.

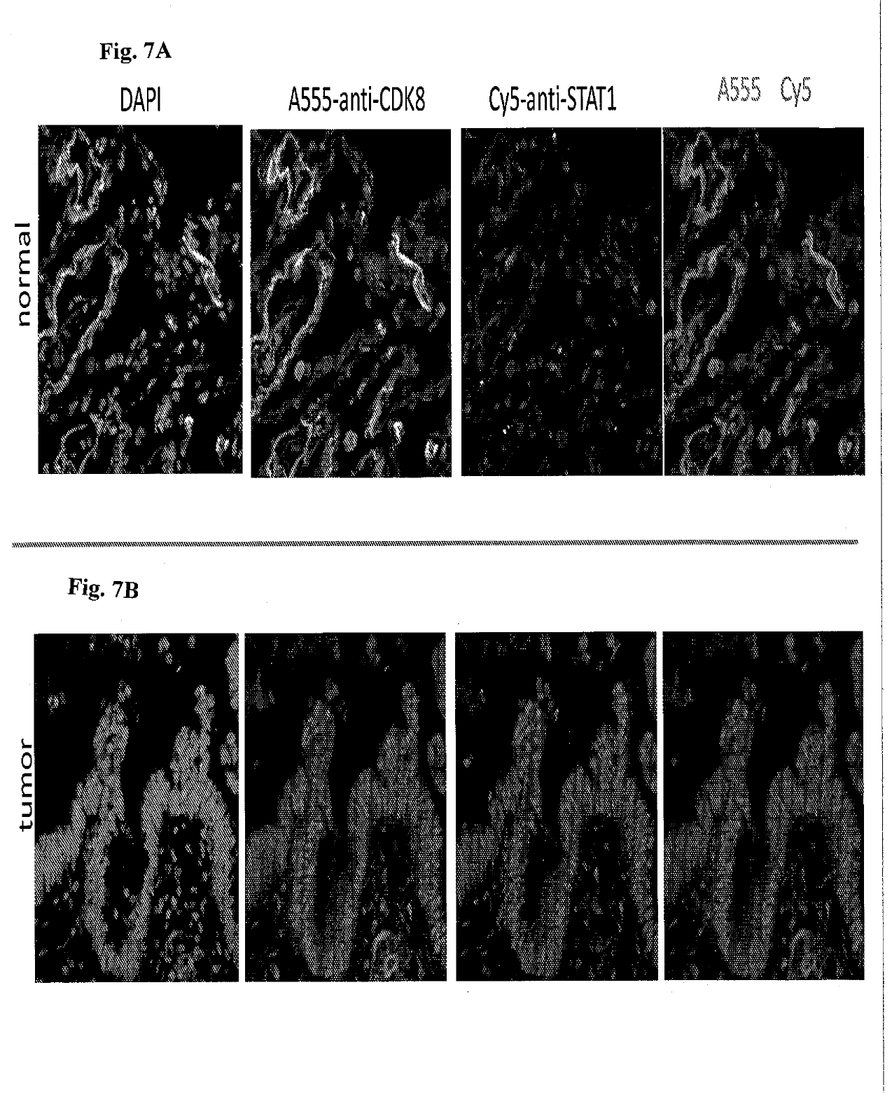

FIG. 7 shows immunofluorescence staining with CDK8 and phospho-STAT1 S727 antibodies of tumor and normal lung tissues of a lung adenocarcinoma. Both CDK8 and phospho-STAT1 S727 are co-increased in lung cancer cells relative to normal lung cells.

### Example 2

### Inhibition of STAT1 S727 Phosphorylation by a CDK8/19 Inhibitor is Involved in CDK8-Mediated Regulation of NFκB Activity

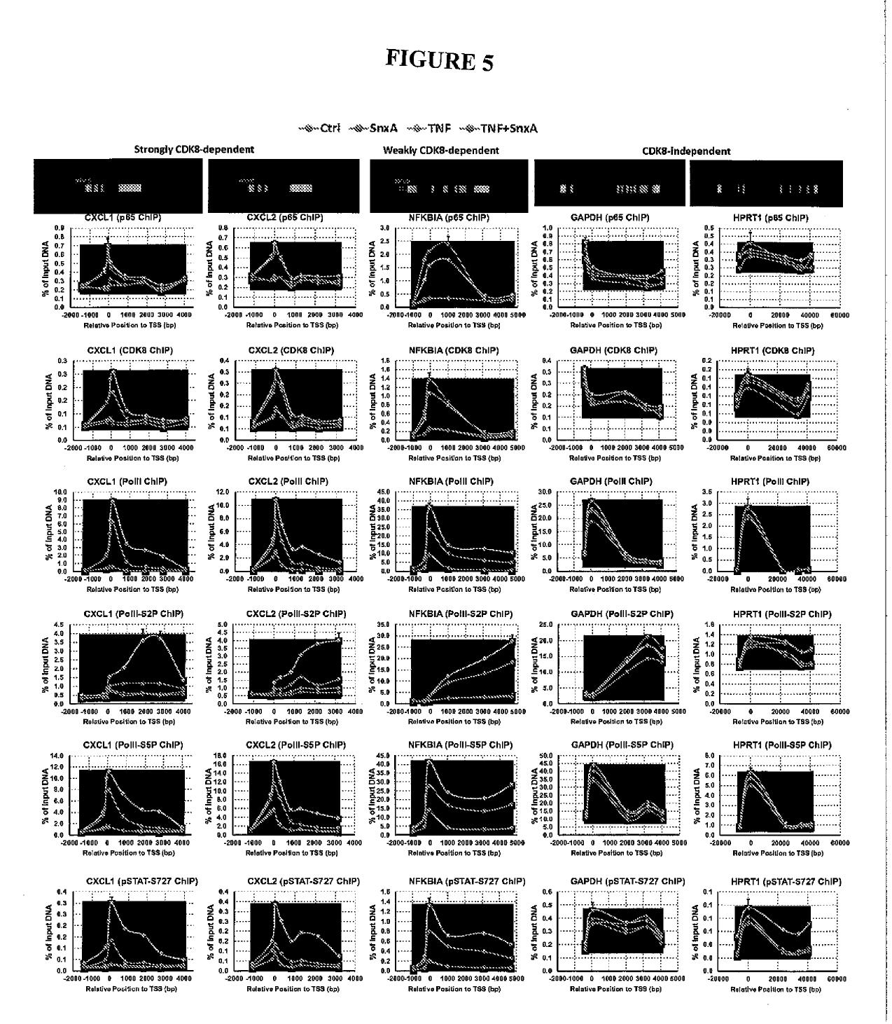

We have previously discovered that CDK8/19 inhibition decreases the induction of transcription via transcription-initiating factor NFκB, upregulation of which is implicated in many different diseases; in particular, CDK8/19 inhibition has a strong effect on the induction of tumor-promoting cytokines CXCL1 and CXCL2 by a canonical NFκB activator TNFα (Patent Publication US2014-0309224). (Burke et al., 2014) have recently shown that STAT1 cooperates with NFκB in the induction of CXCL1 and CXCL2 transcription by IL-1β in rat insulinoma cells. We have therefore tested whether TNFα-induced CXCL1 and CXCL2 transcription in HEK-293 cells involves STAT1 and whether pSTAT1 in this system is affected by CDK8/19 inhibition. This analysis was conducted by chromatin immunoprecipitation (ChIP) of 293 cells that were pre-treated with 5 μM CDK8/19 inhibitor Senexin A (Porter et al., 2012) or carrier for one hour, at which point either TNFα at 10 ng/ml or carrier was added for 30 min. Sonicated chromatin was extracted as described (Galbraith et al., 2013) and immunoprecipitated with the antibodies against the indicated proteins. The ChIP preps were then used for quantitative real-time PCR amplification of different segments of the genes that are strongly induced by TNFα and the induction of which is strongly (˜10-fold) inhibited by Senexin A (CXCL1 and CXCL2), one gene that is strongly induced by TNFα and the induction of which is weakly (˜1.4-fold) inhibited by Senexin A (NFKBIA), and two housekeeping genes that are not regulated by TNFα or Senexin A (HGPRT and GAPDH). The results of this analysis for all four conditions (untreated, Senexin A, TNFα, and TNFα plus Senexin A) are shown in FIG. 5, with the genomic positions of the tested ChIP primers diagrammed at the top of the figure.

The addition of TNFα recruited both the p65 subunit of NFκB and CDK8 to the promoters of the NFκB-regulated but not the housekeeping genes; CDK8 kinase activity inhibition by Senexin A had little or no effect on the recruitment of these proteins. TNFα treatment also recruited Pol II to the regulated genes, and Senexin A had a moderate effect on Pol II recruitment and its distribution along the regulated genes. Senexin A strongly inhibited Pol II CTD phosphorylation at the strongly regulated genes and to a lesser extent at the weakly regulated gene, decreasing both S5P and especially S2P phosphorylation of the CTD, which is consistent with the regulatory role of CDK8 in Pol II CTD phosphorylation of the NFκB-induced genes. Remarkably, similar results (a strong effect on strongly regulated CXCL1 and a weaker effect on NFKBIA, with little or no effect on the housekeeping genes) were obtained for phospho-STAT1 S727 (pSTAT1) (FIG. 5). This result indicates that CDK8/19 regulates NFκB-initiated transcription through phosphorylation of both STAT1 S727 and Pol II CTD.

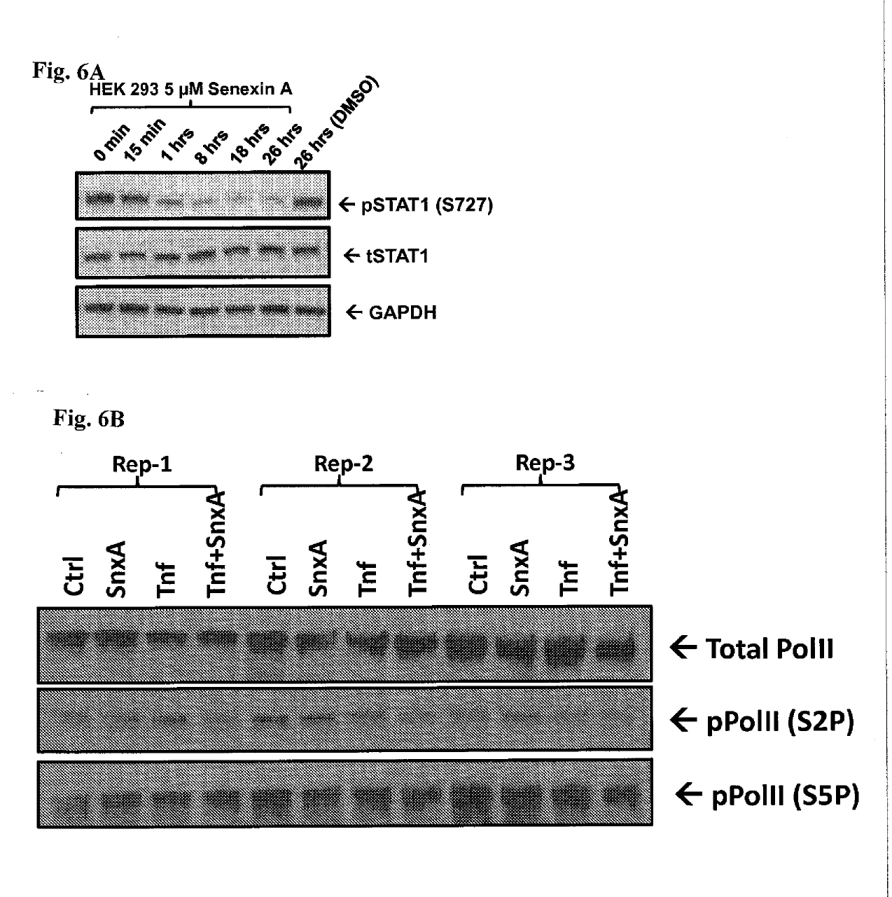

STAT1 S727 phosphorylation is strongly inhibited by 5 μM Senexin A in the whole-cell protein extracts of 293 assay (FIG. 6A), with the effect becoming noticeable even after 15 min and very strong after 1 hr of treatment. In contrast, total Pol II CTD phosphorylation is not significantly affected by Senexin A, as shown by immunoblotting of whole-cell lysates of 293 cells (FIG. 6B). Hence, STAT1 S727 phosphorylation is not only a marker for CDK8/19 activity but also a mechanistic determinant of its essential effects.

The ChIP analysis in FIG. 5 also shows that the association of pSTAT1 with any of the tested genes in the absence of TNFα was unaffected by Senexin A (total of 90 min treatment), even though the level of pSTAT1 in whole-cell lysates is strongly decreased by Senexin A even after 60 min (FIG. 6A). This result suggests that DNA-bound STAT1 is selectively resistant to dephosphorylation under the conditions of CDK8/19 inhibition, thereby offering an explanation why CDK8/19 inhibition does not fully inhibit STAT1 S727 phosphorylation.

What is claimed is:

1. A method for determining the efficacy of a small molecule for inhibiting one or more of cyclin-dependent kinase 8 (CDK8) and cyclin-dependent kinase 19 (CDK19), the method comprising obtaining, from a subject that has been administered the small molecule and has not been administered interferon-γ, a tissue sample or a sample derived from a tissue sample, other than natural killer cells, assaying the sample for signal transducer and activator of transcription protein 1 (STAT1) that is phosphorylated at serine 727 (pSTAT1), quantitating the amount of pSTAT1 in the sample, comparing the amount of pSTAT1 in the sample with an amount of pSTAT1 in a standard, and determining that the small molecule has efficacy for inhibiting CDK8/19 if the amount of pSTAT1 in the sample is statistically significantly lower than the amount of pSTAT1 in the standard.

2. The method according to claim 1, wherein the subject is a mammal.

3. The method according to claim 2, wherein the mammal is a human.

4. The method according to claim 1, wherein the mammal is a rodent.

5. The method according to claim 1, wherein the tissue sample contains or is derived from one or more cell types selected from the group consisting of cancer cells, PBMCs, hair follicle cells, platelets, skin cells, and oral buccal cells.

6. The method according to claim 1, wherein the sample is assayed from about 30 minutes to about 48 hours after the subject has been administered the small molecule.

7. The method according to claim 1, wherein the sample is in situ.

8. The method according to claim 1, wherein the assay is selected from the group consisting of immunoblotting, ELISA, flow cytometry, immunofluorescence, immunohistochemistry and non-antibody based protein detection methods.

9. The method according to claim 1, wherein the standard is a tissue sample obtained from the subject prior to administration of the small molecule.

10. The method according to claim 1, wherein the sample derived from the tissue sample is selected from the group consisting of a whole cell lysate and a nucleoplasmic fraction separated from a chromatin fraction.

11. A method for determining the efficacy of a small molecule for inhibiting one or more of cyclin-dependent kinase 8 (CDK8) and cyclin-dependent kinase 19 (CDK19), the method comprising contacting a sample of cultured cells with the small molecule, other than natural killer cells, assaying the sample for signal transducer and activator of transcription protein 1 (STAT1) that is phosphorylated at serine 727 (pSTAT1), quantitating the amount of pSTAT1 in the sample, comparing the amount of pSTAT1 in the sample with an amount of pSTAT1 in a standard, and determining that the small molecule has efficacy for inhibiting CDK8 if the amount of pSTAT1 in the sample is statistically significantly lower than the amount of pSTAT1 in the standard.

12. The method according to claim 11, wherein the sample is selected from the group consisting of a whole cell lysate and a nucleoplasmic fraction separated from a chromatin fraction.
